(19) United States
(12) Patent Application Publication
(10) Pub. No.: US 2023/0293742 A1
(43) Pub. Date: Sep. 21, 2023

(54) SYSTEMS AND METHODS FOR ELECTRO-MECHANICAL UV STERILIZATION

(71) Applicant: HARBOR INNOVATIONS, LLC, Oyster Bay, NY (US)

(72) Inventors: Richard J. KOCINSKI, Punta Gorda, FL (US); Erick E. RIOS, New Haven, CT (US); Brian A. Wong SHUI, Atlanta, GA (US); Robert Paul BASIL, Lawrenceville, GA (US); Devin Lee MOORE, Decatur, GA (US)

(21) Appl. No.: 18/021,011

(22) PCT Filed: Aug. 13, 2021
(86) PCT No.: PCT/US2021/045964

§ 371 Date: Feb. 13, 2023

Related U.S. Application Data
(60) Provisional application No. 63/065,237, filed on Aug. 13, 2020.

(51) Int. Cl. A61L 2/10; A61L 2/24

(52) U.S. Cl. CPC A61L 2/10; A61L 2/24; A61L 2202/11; A61L 2202/14

(57) ABSTRACT

An apparatus for destroying pathogens includes a housing, a platform, and a sanitizing unit. The platform has a window configured to permit passage of ultraviolet light therethrough. The housing is configured to receive the platform. The sanitizing unit is disposed below the platform and includes a track assembly, at least one light bar coupled to the track assembly and a motor assembly configured to move the light bar along the track assembly. The light bar includes a first ultraviolet (UV) light emitting source configured to emit UV light towards the platform.

### CROSS-REFERENCE TO RELATED APPLICATION

This application claims the benefit of U.S. Provisional Patent Application No. 63/065,237, filed on Aug. 13, 2020, entitled “SYSTEMS AND METHODS FOR ELECTRO-MECHANICAL UV STERILIZATION,” the entire contents of which are incorporated herein by reference.

### TECHNICAL FIELD

The present disclosure relates to devices for cleaning small and medium sized objects, such as footwear. More specifically, the present disclosure relates to apparatuses and devices that uses ultraviolet light to destroy or inhibit the growth of surface pathogens, such as, for example, virus, bacteria, mold, spores, and fungi, and/or to reduce chemical contaminants.

### BACKGROUND

The soles of footwear are a primary vehicle for pathogens entering homes and healthcare facilities. The pathogens can cause sickness, disease, and possible death. Door mats, the primary means for cleaning shoe bottoms, remove dirt but do not kill pathogens and therefore can quickly become an incubator for germs. Other solutions such as liquid dips are not practical for high traffic areas and require frequent maintenance to stay effective. Disposable booties or shoe covers are used in professional environments, such as sterile surgical theaters, but do not work well in public areas, as people tend to be self-conscience about wearing them, and there are safety concerns over people tripping while wearing such covers. Accordingly, there is a need for improved devices and methods for sanitizing footwear.

### SUMMARY

This disclosure relates to systems and methods for destroying pathogens on an object using ultraviolet (UV) light. An apparatus for destroying pathogens includes a housing and a platform having a window configured to permit passage of ultraviolet light therethrough. The platform is configured to be received by the housing. A sanitizing unit is disposed below the platform, and includes: a track assembly, a light bar coupled to the track assembly, and a motor assembly configured to move the light bar along the track assembly. The light bar includes a first UV light emitting source and configured to emit UV light towards the platform.

In aspects, the apparatus may further include a controller having a processor and a memory with instructions stored thereon, the instructions, which, when executed by the processor, cause the motor to move the light bar along the track assembly.

In aspects, the light bar may further include a second UV light-emitting source and a first sensor configured to detect which of the first and second UV light emitting sources are approximately below an object on the platform.

In aspects, the controller may be configured to cause at least one of the first and second UV light emitting sources to emit UV light when the sensor detects the at least one of the first and second UV light emitting sources to be approximately below the object.

In aspects, the light bar may further include a plurality of UV light emitting sources that includes the first UV light emitting source.

In aspects, the motor assembly may cause the light bar to move along the track assembly at a first rate of travel and a second rate of travel slower than the first rate of travel.

In aspects, the light bar may be configured to travel along the track at the first rate of travel when the light bar is not approximately below an object on the platform and the light bar may be configured to travel at the second rate of travel when the light bar is approximately below the object on the platform.

In aspects, the track assembly may include a sliding mechanism configured to couple the light bar to the track assembly.

In aspects, the light bar may include a plurality of sensors, each sensor configured to detect if the first UV light source is below an object on the platform.

In aspects, the window of the platform may be comprised of at least one of: fused silica, quartz glass, or sapphire.

In aspects, the housing may include an alcove.

In aspects, the apparatus may include at least one reflective surface configured to reflect UV light.

This application discloses another aspect of an apparatus for destroying pathogens that includes: a housing configured to receive an object to be sanitized; at least one light source configured to emit UV light towards the object, the light source disposed in the housing; and a motor coupled to the light source and configured to at least one of a translation or rotation of the light source.

In aspects, the housing may include a bottom housing and a top housing which when coupled together define a cavity configured to receive the light source.

In aspects, the light source may be configured to rotate about an axis.

In aspects, the housing may be configured to support the weight of a person.

In aspects, the light source may span a width of the housing may be configured to be moveable along a length of the housing.

In accordance with yet another aspect of this disclosure, a method of sanitizing an object includes: detecting an object on a platform, the platform configured to permit passage of ultraviolet light therethrough; moving a light bar disposed below the platform along a track assembly, the light bar having a plurality of UV light sources; and emitting UV light from the light bar to destroy pathogens on the object on the platform.

In aspects, the method may include detecting which UV light sources of the plurality of UV lights sources are approximately below the object and selectively emitting UV light from the UV light sources approximately below the object.

### BRIEF DESCRIPTION OF THE DRAWINGS

The accompanying drawings, which are incorporated in and constitute a part of this specification, illustrate aspects and features of the disclosure and, together with the detailed description below, serve to further explain the disclosure, in which:

FIG. 1 is a perspective view of a sanitizing system according to an aspect of the present disclosure;

FIG. 2 is an exploded view of the sanitizing system of FIG. 1 showing a track assembly, a LED light bar, and a housing according to the present disclosure;

FIG. 3 is a perspective view of the track assembly and LED light bar disposed in the housing of the sanitizing system of FIG. 2;

FIG. 4 is a perspective view of another light bar and track assembly in accordance with another aspect of this disclosure;

FIG. 5 is a sectional view of the sanitizing system of FIG. 1 taken along lines 5-5 on FIG. 3;

FIG. 6 is a perspective view of another sanitizing system, in accordance with another aspect of this disclosure;

FIG. 7 is a perspective view of yet another sanitizing system in accordance with yet another aspect of this disclosure;

FIG. 8 is a cross-sectional view of the sanitizing system of FIG. 7; and

FIG. 9 is a diagram of a method for sanitizing an object according to the present disclosure.

Further details and various aspects of this disclosure are described in more detail below with reference to the appended figures.

### DETAILED DESCRIPTION

Aspects of the presently disclosed systems and methods for electro-mechanical ultraviolet (UV) light sterilization are described in detail with reference to the drawings, in which like reference numerals designate identical or corresponding elements in each of the several views. However, it is to be understood that the disclosed devices are merely exemplary of the disclosure and may be embodied in various forms. Well-known functions or constructions are not described in detail to avoid obscuring the disclosure in unnecessary detail. Therefore, specific structural and functional details disclosed herein are not to be interpreted as limiting, but merely as a basis for the claims and as a representative basis for teaching one skilled in the art to variously employ the disclosure in virtually any appropriately detailed structure. While reference to a sanitizing system configured to sanitize footwear is made herein, it is envisioned that the sanitizing system be operable to sanitize a variety of objects not specifically disclosed by the present disclosure as can be envisioned by those of ordinary skill in the art.

As used herein, the term “pathogens” includes, but is not limited to, viruses, coronaviruses (e.g. COVID-19), bacteria (e.g., Staphylococcus aureus, MRSA, CDIF, VRE, Pseudomonas aeruginosa and E. coli), molds, spores, fungi, or the like.

The present disclosure provides a sanitizing system with electro-mechanical means that utilizes ultraviolet (UV) light emitting devices to sanitize objects, such as footwear, in a variety of settings including, but not limited to, hospitals, homes, laboratories, etc. In aspects, UV light emitting devices, such as UV bulbs, UV light emitting diodes (LEDs), or UV IR (infrared) sources may emit light directed towards an object via a variety of electromechanical means including, but not limited to, UV light emitting devices moveably coupled to a track, rotating mirrors to direct UV light towards an object, or UV light emitting devices coupled to rotary or wiper systems for passing UV light around an object as described in further detail below.

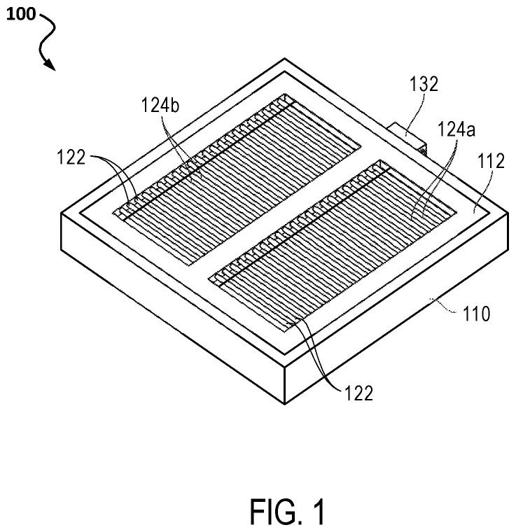

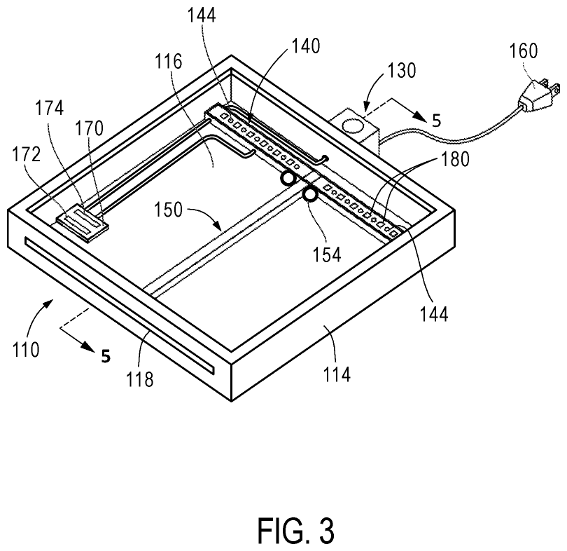

With reference to FIGS. 1-5, a sanitizing system 100 configured to sanitize an object (e.g., footwear) includes a housing 110, a platform 120, a motor assembly 130, a light bar 140, and a track assembly 150. The motor assembly 130, the light bar 140, and the track assembly 150 define an electro-mechanical sanitizing unit. The housing 110 includes a top housing 112 configured to receive the platform 120 and a bottom housing 114 configured to couple to the top housing 112, the top and bottom housing 112, 114, when coupled together define an internal cavity 116. The light bar 140 includes UV light sources 144 such as UV LEDs or a UV bulb (not shown). The light bar 140 is coupled to the motor assembly 130 and track assembly 150. The motor assembly 130, light bar 140, and track assembly 150 are disposed in the internal cavity 116 of the housing 110 and below the platform 120.

A power supply 160, controller 170, and/or sensors 180 are in electrical communication with the motor assembly 130, light bar 140, and/or track assembly 150. The controller 170 and/or sensors 180 may be disposed within the internal cavity 116 of the housing 110. The controller 170 includes a processor 174 and a computer memory 172 with instructions stored thereon, which, when executed by the processor 174, causes the motor assembly 130 to move the light bar 140 along the track assembly 150 and causes the light bar 140 to emit UV light for destroying pathogens on footwear placed on the platform 120 of the sanitizing system 100.

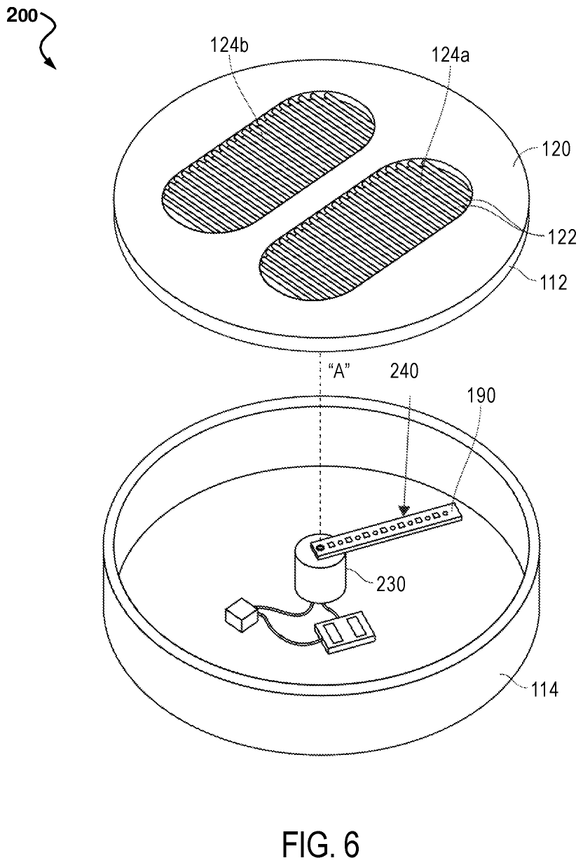

The housing 110 may define a square shape. In some aspects, the housing 110 may define a rectangular, circular (see FIG. 6), trapezoidal, or any suitable shape. The platform 120 defines one or more windows and may include a frame 122 and a pair of plates 124a, 124b supported by the frame 122. The frame 122 and the housing 110 may be fabricated from a non-corrosive material that prevents UV light therethrough. In aspects, the frame 122 and the housing 110 may be fabricated from any suitable material such as various metals, including aluminum, or suitable plastics.

The plates 124a, 124b are elongated and sized to accommodate any size foot of a person standing on the platform 120. The plates 124a, 124b are shown as rectangular in shape, but it is contemplated the plates 124a, 124b may assume any suitable shape dimensioned to accommodate an entire foot of a person. For example, the plates 124a, 124b may be shaped as an oversized footprint. The plates 124a, 124b are received by the frame 122 of the platform 120 and are formed from a material (such as quartz glass) that permits passage of the UV-C light therethrough while also exhibiting strength to support the weight of a person. The plates 124a, 124b may be fabricated from materials such as fused silica, quartz glass, sapphire, or the like known by those of ordinary skill in the art to permit passage of UV light therethrough. The plates 124a, 124b are configured to operate as the one or more windows through which UV light may be transmitted.

The light bar 140 may be configured to emit short-wavelength UV (UV-C) light. The light bar 140 may include UV LEDs, UV light bulbs, germicidal light bulbs, or UV lasers. The light bar 140 may include UV LEDs 144, such as UV-C LEDs to form a LED light bar. In other aspects, the light bar 140 includes a UV-C light bulb. The UV LEDs may be configured to emit other wavelengths of ultraviolet light instead of or in addition to UV-C light. UV-C LEDs, which are smaller than standard UV-C light bulbs, allow for a miniaturization of the overall footwear-sanitizing system and a longer use life. The UV-C LEDs are disposed on a printed circuit board 146 forming the light bar 140. The LED light bar 140 is coupled to a motor assembly 130 and a track assembly 150 to permit movement of the LED light bar 140 over an area. The light bar 140 may be coupled to a track assembly 150, such that when activated, the light bar 140 travels along the track assembly 150 and emits UV light directed towards the platform 120 for destroying or inactivating pathogens on the footwear. Each of the UV LEDs 144 disposed on the light bar 140 may be discreetly controlled such that only those UV LEDs 144 directly below the feet of a user on the platform 120 will be activated as the light bar 140 passes beneath the feet of a user, or as it passes beneath an object.

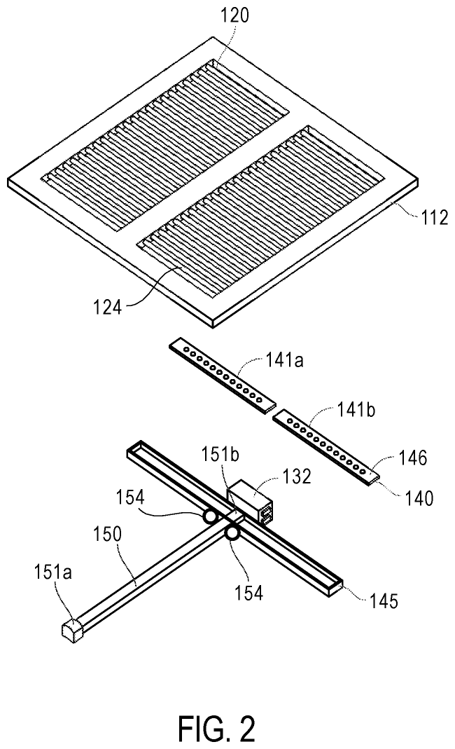

In aspects, the light bar 140 is perpendicular to a track assembly 150 disposed down the center of the sanitizing system 100. As shown in FIG. 2, the light bar 140 may include two sections 141a, 141b, each section spanning at least the width of a plate 124a of the platform 120. The light bar 140, when positioned at an end nearest the housing 110, may form a “T” with the track assembly 150. In other aspects, the light bar 140 may be coupled at one end to the track assembly 150, such that when the light bar 140 is positioned at one of the ends 151a, 151b of the track assembly nearest the housing 110 an “L” is formed. In further aspects, the track assembly 150 is adjacent to and extends the length of a side of the housing 110 such that the light bar 140 and the tack assembly 150 form the “L.”

The light bar 140 may include a structural support 145 in the form of a plate, bar, or other well-known structural support. The printed circuit board 146 with UV LEDs 144 may be rigidly fastened to the structural support 145. The light bar structural support 145 may be manufactured from plastic, metal, and/or other known materials. The light bar 140 may include UV-LEDs arranged longitudinally on a printed circuit board 146 of the light bar 140.

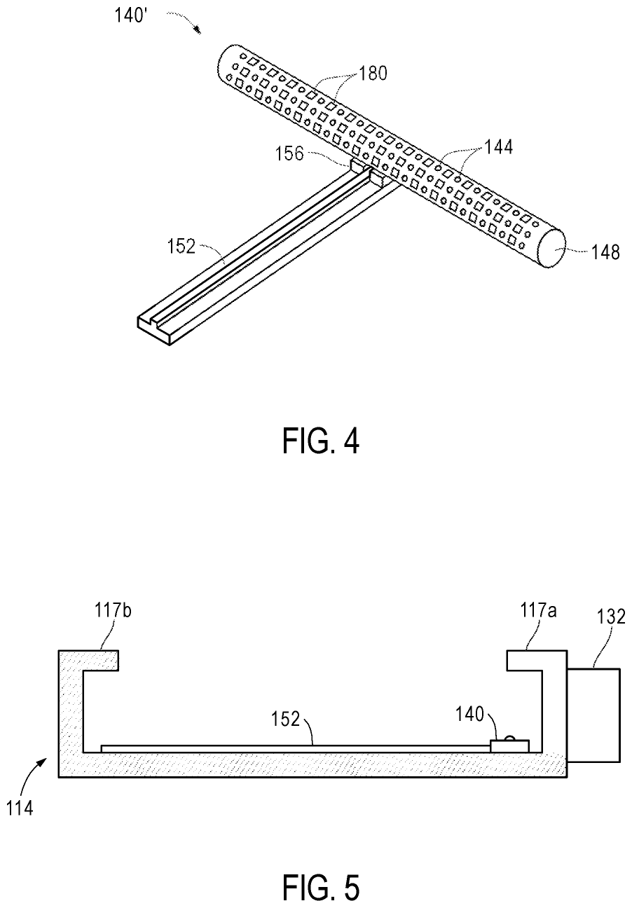

Referring to FIG. 4, the sanitizing system 100 may include a light bar 140′ that may include one or more rows of UV LEDs 144. The light bar 140′ include the structural support 145 which may be a rod 148. The UV LEDs 144 are arranged along one half of the rod 148 and facing the platform 120. In this manner, as the light bar 140′ passes beneath the footwear of a user, the footwear may be sanitized in longer amounts while maintaining the same amount of time at which the light bar 140 will spend traveling below the footwear.

The light bar 140 may include reflective surfaces 190 for directing the UV light emitted by the light bar 140 towards the platform 120. The reflective surfaces 190 may include white polytetrafluorethylene coatings, mirrors, or other reflective surfaces well known by those of ordinary skill in the art. In some aspects, the housing 110 may be comprised of reflective surfaces 190 for directing the UV light emitted by the light bar 140 towards the platform 120. The housing 110 may include a tray 118 with reflective surfaces 190 for directing the UV light emitted by the light bar 140 towards the platform 120.

With continuing reference to FIGS. 1-4, the light bar 140 (or light bar 140′) may be coupled to the track assembly 150 via wheels 154 disposed on a track 152, the wheels 154 coupled to a motor assembly 130 configured to actuate the wheels 154 to permit travel of the light bar 140 along the track assembly 150. The track assembly 150 may be a linear motion track assembly 150, and the light bar 140 may be coupled to a sliding mechanism 156 (see FIG. 4) of the linear motion track assembly 150. The sliding mechanism 156 may be linear bearings, rolling bearings, rollers, slides or other well-known sliding mechanisms. The light bar 140 may be magnetically coupled to the track assembly 150 by magnetic levitation and configured to move along the track assembly 150 via electromagnetic forces exerted by a motor assembly 130 and controller 170. The light bar 140 may be coupled to the track assembly 150 via roller bearings, track rollers, telescoping slides, linear bearings, positioning slides and/or other motion track assemblies known by those of ordinary skill in the art. It is envisioned that any suitable track assembly be used, such as a circular track assembly. In aspects, the light bar 140 may be configured to change angles at different portions of the track assembly 150.

Returning to FIGS. 1-4, the motor assembly 130, track assembly 150 and/or the light bar 140 are in electrical communication with a controller 170. The motor assembly 130 permits the light bar 140 to travel along the track assembly 150, the track assembly 150 generally spanning the length or width of the platform 120. In this manner, the light bar 140 is permitted to sanitize the footwear of a user. The controller 170 may be configured to control the motor 132 and/or the UV LEDs 144 or a UV light bulb (not shown) of the light bar 140. The controller 170 includes a processor and computer memory with instructions stored thereon, which when executed by the processor, activates the motor and causes the light bar 140 to travel the length of the track assembly 150. The controller 170 may be coupled to sensors 180 that detect the presence of the footwear of a user on the platform 120. The sensors 180 may be part of the housing 110, platform 120, and/or the light bar 140. In aspects, the light bar 140 may include sensors 180 in electrical communication with the controller 170, the sensors 180 configured to detect footwear or an object on the platform 120 or absence thereof as the light bar 140 travels along the track assembly 150. When the sensors 180 detect no footwear or object above the light bar 140, the controller 170 may speed up the rate at which the light bar 140 travels along the track assembly 150 until the light bar 140 is approximately below the footwear or object on the platform 120. When the sensors 180 detect footwear or an object above the light bar 140 on the platform 120, the controller 170 may slow the rate at which the light bar 140 travels along the track assembly 150 to a rate to allow the UV light emitted by the light bar 140 to sanitize the footwear. By controlling the rate of travel of the light bar 140, the total time the user must spend on the platform 120 in order to sanitize his or her footwear may be reduced. For example, if a user with very small feet/footwear is standing on the platform 120 near an edge of the housing 110 opposite the location of the light bar 140, the motor assembly 130, when activated by the controller 170, may cause the light bar 140 to travel at a faster rate beneath the uncovered portions of the platform 120 until the light bar 140 reaches the user's feet. Once the light bar 140 senses it is under the covered portion of the platform 120, (the user's feet/footwear), the rate of travel of the light bar 140 along the track assembly 150 is slowed. The light bar 140 may move at a rate from about zero (0) inches per second (in/s) (about 1 mm/s) to about twelve (12) in/s (about 305 mm/s). When the light bar 140 is not approximately beneath footwear the rate of travel of the light bar 140 may be equal to or greater than one (1) in/s (about twenty-five (25) mm/s). When the light bar 140 is approximately beneath footwear, the rate of travel of the light bar 140 may be from about half (0.5) an in/s (twelve (12) mm/s) to about one (1) in/s (about twenty-five (25) mm/s). In some aspects, the rate may vary between twenty-five 25 mm/s to about seventy (70) mm/s depending on whether the light bar 140 is under footwear or not. The rate may be determined depending on the length of the footwear such that the footwear is subjected to UV light for enough time to inactivate pathogens thereon. It is contemplated that the light bar 140 may make multiple passes at a high rate to achieve the same dose of UV light as if it had a slower rate.

In further aspects, UV LEDs 144 disposed on the light bar 140 may be discreetly controlled so that only the UV LEDs 144 of the light bar 140 directly beneath the footwear of a user or object thereon are activated to emit UV light. For example, a user with very narrow feet/footwear may be detected by the sensors 180 of the light bar 140, and the controller 170 then discretely controls each individual UV LED 144 on the light bar 140 such that only those beneath the user's feet/footwear emit UV light to destroy pathogens on the user's footwear. In another example, a user with wider feet/footwear may be detected by the sensors 180 of the light bar 140 such that the controller 170 then discretely controls more UV LEDs 144 that are under the user's feet/footwear versus the user with narrower feet. In aspects, each UV LED 144 may be configured to emit light only when below the footwear of a user or below any other object.

Additionally, when the light bar 140 is a rod with UV LEDs 144 disposed around the top half of the rod 148 facing the platform 120, the UV LEDs 144 angled towards the footwear on the platform 120 may be discretely controlled to emit UV light directed towards parts of the footwear not directly above the light bar 140 (see FIG. 5). For example, as the light bar 140 travels towards the footwear of the user, those UV LEDs 144 disposed and angled in the direction of travel (forward UV LEDs) may be first controlled to emit UV light directed towards the footwear. As the light bar 140 passes under the user's footwear, the UV LEDs 144 directly under and facing upwards (upward UV LEDs) and the UV LEDs facing opposite the direction of travel (rearward UV LEDs) may be turned on so as to lengthen the amount of sanitization time of the parts of the footwear that have been passed by the light bar 140. As the light bar 140 approaches the end of the user's footwear, the forward UV LEDs 144 may be discretely controlled to turn off such that only the rearward UV LEDs 144 and upward UV LEDs 144 emit UV-light. This allows the sanitizing system 100 to efficiently destroy the pathogens on the footwear of the user. In aspects, each UVC-LED may have a sensor to detect the presence of a surface, and a processor or controller includes logic that will only illuminate the LED when a surface is present. This minimizes UV-C light from escaping around an item to be disinfected. By selecting the LEDs to be activated, the system is kept cooler which may save on battery life (if connected to the system) and/or power utilization. Additionally, this may prolong the life of the UV light sources 144, and prevents unnecessary UV radiation.

Referring to FIG. 5, the housing 110 may be configured to include a first alcove 117a where the light bar 140 may be parked when not actively sanitizing footwear or an object on the platform 120. When the controller 170 and sensors 180 thereof detect a user's footwear on the platform 120, the motor 132 will cause the light bar 140 to travel out of the first alcove 117a. When the controller 170 and sensors 180 detect that the light bar 140 has completely passed below and sanitized the footwear on the platform 120, the controller 170 may cause the motor assembly 130 to return the light bar 140 to the first alcove 117a. In aspects, the housing 110 may be configured to include a second alcove 117b, the first and second alcoves 117a, 117b on opposite ends of the track assembly 150 so that the system does not need to return the light bar 140 to whichever of the first and second alcoves 117a, 117b it was previously positioned. This allows bi-directional use of the sanitizing system 100. In this case, if the light bar 140 is a rod 148, the forward UV LEDs and rearward UV LEDs may switch roles, the forward UV LEDs becoming the rearward UV LEDs and vice versa.

In aspects, multiple track assemblies 150 may each be coupled to one or more separate light bars 140 disposed in the housing 110 and coupled to one or more motor assemblies 130. The controller 170 may cause one or more separate light bars 140 to travel along their respective track assemblies 150.

The sanitizing system 100 is configured to be installed in or on a floor. For example, the sanitizing system 100 may be configured to be installed as a floor tile or as a doormat. When the sanitizing system 100 is installed in or on the floor in a footwear sanitizing configuration, the LED light bar 140 is configured to direct ultraviolet light upwardly through the platform 120 and onto the bottom of the footwear of the user to destroy pathogens on the user's footwear.

Use of a light bar 140 with UV LEDs provides significant cost reductions versus a sanitizing system with stationary UV light sources (e.g., UV LEDs, UV bulbs, UV lasers). The light bar 140, motor assembly 130, and track assembly 150 allow for full UV LED coverage of an area using significantly less UV LEDs or other UV light sources as would otherwise be necessary.

With reference to FIG. 6, a sanitizing system 200 includes the housing 110, the platform 120, a motor assembly 230, and a light bar 240. The sanitizing system 200 is similar to and includes the features of the sanitizing system 100 and for the sake of brevity only the differences are discussed below. The light bar 240 may be pivotably coupled to a motor assembly 230. The light bar 240 is configured to rotate about an axis “A” at the center of the platform 120. The motor assembly 230 is positioned at the center of the internal cavity 116 of the housing 110 such that the motor assembly 230 is configured to rotate the light bar 240 to cover a circular area.

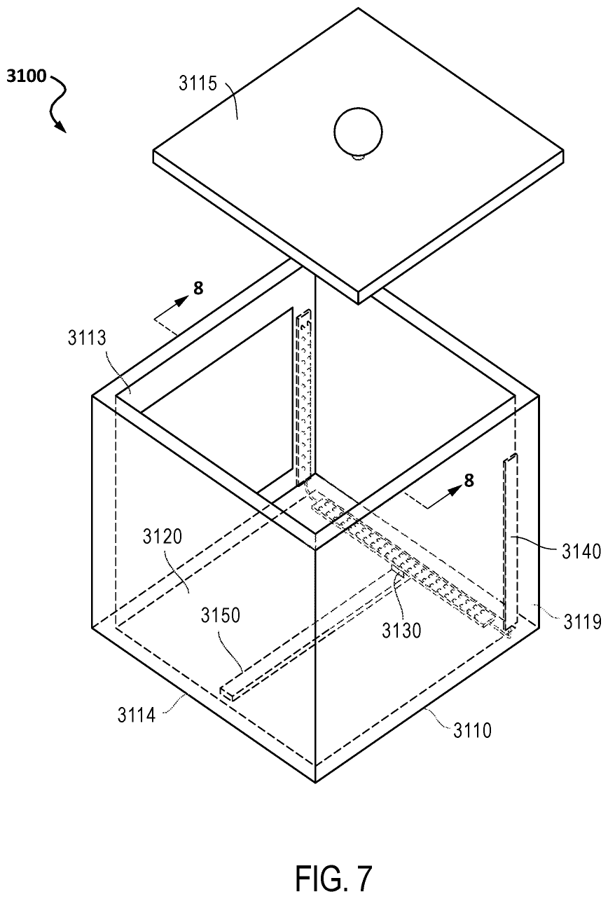

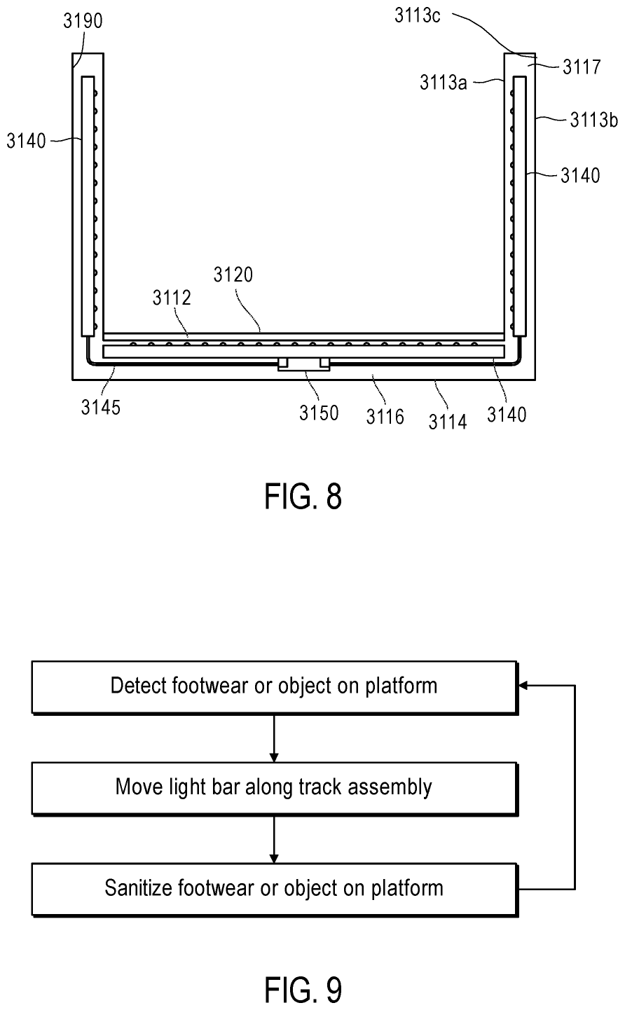

Referring now to FIGS. 7-8, another illustrative sanitizing system 3100 for UV sterilization along a track having a box configuration in accordance with the present disclosure is illustrated. Sanitizing system 3100 is similar to sanitizing system 100, and only the differences are described in detail below. The box configuration includes a housing 3110, a platform 3120, and one or more light bars 3140, one or more motor assemblies 3130, and one or more track assemblies 3150. The housing 3110 includes a cover 3115, a top housing 3112 configured to receive the platform 3120 and a bottom housing 3114 configured to couple to the top housing 3112, the top and bottom housing 3112, 3114 when coupled together define an internal cavity 3116. The top housing 3112 may include walls 3113 extending therefrom configured to receive the cover 3115. The walls 3113 may include reflective surfaces for directing UV light toward an object placed on the platform 3120.

In the box configuration, the sanitizing system 3100 would at least include the features and aspects with respect to the controller, motor assembly, track assembly and light bar described above and will not be described again.

In some aspects, the top and bottom housing 3114, 3112 may include inner walls 3113a and outer walls 3113b that further define internal wall cavities 3117 that are open to the internal cavity 3116 below the platform 3120 and thereby defining a box 3119. The inner wall cavities 3117 are configured to permit UV light into the box 3119, and the inside face 3113c of the outer wall 3113b may have reflective surfaces 3190.

The light bar 3140 may be “U” shaped such that the light bar 3140 is disposed below the platform 3120 and in the internal wall cavities 3117. When in operation, the “U” shaped light bar 3140 is capable of sanitizing from below the platform 3120 and from the walls 3113 of the box 3119. The UV light emitted by the light bars 3140 may be reflected by reflective surfaces on the bottom of the cover 3115 to sanitize all surfaces of an object placed into the box 3119.

In aspects, the one or more track assemblies 3150, the one or motor assemblies 3130, and the one or more light bars 3140 may each be disposed in each wall cavity 3117 and internal cavity 3116 of the housing 3110. In aspects, each track assembly 3150 may be coupled to a respective light bar 3140 and a respective motor assembly 3140. In aspects, a plurality of light bars 3140 may be coupled to each other via a structural support 3145, and the one of the light bars 3140 of the plurality 3140 may be coupled to a track assembly 3150.

It is envisioned that multiple shaped track assemblies may be used, such as L shaped track assemblies each reflected about a diagonal axis defined by two opposite corners of the box. In aspects, the cover 3115 may also include a motor assembly 3130, track assembly 3150, and light bar 3140. It is envisioned that the light bars, track assemblies, and motor assemblies be disposed within the internal wall cavities to protect those components. Alternatively, the light bars, track assemblies, and motor assemblies may be disposed and exposed along the inside walls of the box.

In other aspects of this disclosure, an electro-mechanical UV sanitizing system (e.g., sanitizing system 100, 200, or 3100) may include one or more each of a light bar or UV light emitting source, a motor, and a housing. One or more UV light bars or light emitting sources may be configured to pivot to produce a wiping motion to sweep over an area to be sanitized. In some aspects, the light bars may be stationary, and mirrors may be coupled to motors. The mirrors may be controlled, via the motors, to change the orientation and/or position of each mirror in order to reflect light in a desired direction.

In aspects, lasers that emit UV light may be controlled to sanitize an item by scanning the item. The lasers may be mounted to a track assembly or a ball and socket assembly, and the direction of a beam of the laser directed to desired areas.

In other aspects, the electromechanical UV sanitizing system includes a track or path for an object to be placed on and whereby the object is passed by the light bar or UV light emitting source. The electro-mechanical UV sanitizing system may include a rotary table for turning an object placed thereon to subject all sides of an object to the UV light source.

It is contemplated that a light bar of this disclosure be configured to follow a wiping motion, disposed on a track assembly, include lasers with controllable scanning motions and beams, include controllable mirrors for directing the light, or otherwise be disposed on a track assembly for controlling the position and/or orientation of an object. The UV light emitting devices may be beneath a platform, in a housing, in or on walls of a housing in a box configuration, on the cover of a box or disposed within the space defined by a box. In aspects, the housing may be a platform, a box, or a chamber. In some aspects, objects may be disposed on a platform in the box or the chamber, or may be hung (such as a shirt by a hanger) inside the box or chamber. The electro-mechanical system may include a manipulator assembly for changing the orientation, position, and/or combinations thereof of an object to be sanitized.

With reference to FIG. 9, a method of sanitizing footwear or an object using the system of this disclosure is shown. The method includes sensing an object, such as footwear, placed on a platform 120; moving a light bar configured to destroy pathogens on the footwear using UV light; and sanitizing the footwear. The method includes determining if footwear, or another object, is disposed on the platform 120. Additionally, a light bar configured to emit UV light thereby destroying pathogens is moved from below one end of the platform 120 to the other along a track assembly. The light bar is passed below the footwear as UV light is radiated towards and sanitizes the footwear. In aspects, the method includes only emitting UV light when the light bar is approximately below the footwear on the platform 120. In aspects, when the light bar reaches the end of the platform 120, or finishes sanitizing the footwear, the user is notified that the sanitization is complete. In other aspects, the UV light includes sensing where above the light bar footwear is located and only emitting UV light from the UV LEDs disposed on the light bar approximately below the footwear.

The systems described herein may also utilize one or more controllers to receive various information and transform the received information to generate an output. The controller may include any type of computing device, computational circuit, or any type of processor or processing circuit capable of executing a series of instructions that are stored in memory. The controller may include multiple processors and/or multicore central processing units (CPUs) and may include any type of processor, such as a microprocessor, digital signal processor, microcontroller, programmable logic device (PLD), field programmable gate array (FPGA), or the like. The controller may also include a memory to store data and/or instructions that, when executed by the one or more processors, cause the one or more processors to perform one or more methods and/or algorithms.

Any of the herein described methods, programs, algorithms or codes may be converted to, or expressed in, a programming language or computer program. The terms “programming language” and “computer program,” as used herein, each include any language used to specify instructions to a computer, and include (but is not limited to) the following languages and their derivatives: Assembler, Basic, Batch files, BCPL, C, C+, C++, Delphi, Fortran, Java, JavaScript, Verilog, VHDL, machine code, operating system command languages, Pascal, Perl, PL1, scripting languages, Visual Basic, metalanguages which themselves specify programs, and all first, second, third, fourth, fifth, or further generation computer languages. Also included are database and other data schemas, and any other metalanguages. No distinction is made between languages which are interpreted, compiled, or use both compiled and interpreted approaches. No distinction is made between compiled and source versions of a program. Thus, reference to a program, where the programming language could exist in more than one state (such as source, compiled, object, or linked) is a reference to any and all such states. Reference to a program may encompass the actual instructions and/or the intent of those instructions.

Persons skilled in the art will understand that the structures and methods specifically described herein and shown in the accompanying figures are non-limiting exemplary embodiments, and that the description, disclosure, and figures should be construed merely as exemplary of particular embodiments. It is to be understood, therefore, that this disclosure is not limited to the precise embodiments described, and that various other changes and modifications may be made by one skilled in the art without departing from the scope or spirit of this disclosure. Additionally, the elements and features shown or described in connection with certain embodiments may be combined with the elements and features of certain other embodiments without departing from the scope of this disclosure, and that such modifications and variations are also included within the scope of this disclosure. Accordingly, the subject matter of this disclosure is not limited by what has been particularly shown and described.

What is claimed is:

1. An apparatus for destroying pathogens, comprising:
a housing;
a platform configured to be received by the housing, the platform comprising a window configured to permit passage of ultraviolet light therethrough; and
a sanitizing unit disposed below the platform including:
a track assembly;
a light bar coupled to the track assembly, the light bar having a first ultraviolet (UV) light emitting source configured to emit UV light towards the platform; and
a motor assembly configured to move the light bar along the track assembly.

2. The apparatus according to claim 1, further comprising a controller including a processor and a memory with instructions stored thereon, the instructions, which, when executed by the processor, cause the motor assembly to move the light bar along the track assembly.

3. The apparatus of claim 2, wherein the light bar further includes:
a second UV light-emitting source; and
a sensor configured to detect which of the first and second UV light emitting sources are approximately below an object on the platform.

4. The apparatus of claim 3, wherein the controller is configured to cause at least one of the first and second UV light emitting sources to emit UV light when the sensor detects the at least one of the first and second UV light emitting sources to be approximately below the object.

5. The apparatus of claim 1, further comprising a plurality of UV light emitting sources that includes the first UV light emitting source.

6. The apparatus of claim 5, wherein the plurality of UV light emitting sources are a plurality of UV light emitting diodes (LEDs).

7. The apparatus of claim 1, wherein the light bar is configured to move along the track assembly at a first rate of travel and a second rate of travel slower than the first rate of travel.

8. The apparatus of claim 7, wherein the light bar is configured to travel along the track assembly at the first rate of travel when the light bar is not approximately below an object on the platform and the light bar is configured to travel at the second rate of travel when the light bar is approximately below the object on the platform.

9. The apparatus of claim 1, wherein the track assembly includes a sliding mechanism configured to couple the light bar to the track assembly.

10. The apparatus of claim 1, wherein the light bar includes a plurality of sensors, each sensor configured to detect if the first UV light source is below an object on the platform.

11. The apparatus of claim 1, wherein the window of the platform is comprised of at least one of: fused silica, quartz glass, or sapphire.

12. The apparatus of claim 1, wherein the housing includes an alcove.

13. The apparatus of claim 1, further including at least one reflective surface configured to reflect UV light.

14. An apparatus for destroying pathogens, comprising:
a housing configured to receive an object to be sanitized;
a light source configured to emit ultraviolet (UV) light towards the object, the light source disposed in the housing; and
a motor coupled to the light source and configured to cause at least one of a translation or rotation of the light source.

15. The apparatus of claim 14, wherein the housing includes a bottom housing and a top housing which when coupled together define a cavity configured to receive the light source.

16. The apparatus of claim 14, wherein the light source is configured to rotate about an axis.

17. The apparatus of claim 14, wherein the housing is configured to support a weight of a person.

18. The apparatus of claim 14, wherein the light source spans a width of the housing, the light source configured to be moveable along a length of the housing.

19. A method of sanitizing an object comprising:
detecting an object on a platform, the platform configured to permit passage of ultraviolet light therethrough;
moving a light bar disposed below the platform along a track assembly, the light bar having a plurality of ultraviolet (UV) light sources; and
emitting UV light from the light bar to destroy pathogens on the object on the platform.

20. The method of claim 19, further comprising, detecting which UV light sources of the plurality of UV lights sources are approximately below the object and selectively emitting UV light from the UV light sources approximately below the object.
